(19) United States
(12) Patent Application Publication
(10) Pub. No.: US 2026/0240723 A1
(43) Pub. Date: Aug. 20, 2026

(54) THERAPEUTIC GARMENT FOR TREATING FLUID OVERLOAD

(71) Applicant: AQUAPASS LTD, Or-Akiva (IL)

(72) Inventors: Yaacov Nitzan, Or-Akiva (IL); Lotem Mahluf Monfred, Ra'anana (IL)

(21) Appl. No.: 19/543,718

(22) Filed: Feb. 18, 2026

Related U.S. Application Data
(60) Provisional application No. 63/759,803, filed on Feb. 18, 2025.

(51) Int. Cl. A61H 35/00; A61F 7/00

(52) U.S. Cl. CPC A61H 35/00; A61F 7/0053; A61F 2007/006; A61F 2007/0093; A61H 2035/004; A61H 2201/1645

(57) ABSTRACT

The invention generally relates to a therapeutic garment for treating fluid overload in a patient by providing controlled stimulation of fluid transfer directly and non-invasively from an interstitial compartment of the patient through the skin of the patient.

### CROSS-REFERENCE TO RELATED APPLICATIONS

This application claims priority to, and the benefit of, U.S. Provisional Application No. 63/759,803, filed Feb. 18, 2025, the content of which is incorporated by reference herein in its entirety.

### FIELD OF THE INVENTION

The invention generally relates to a therapeutic garment for treating fluid overload in a patient by providing controlled stimulation of fluid transfer directly and non-invasively from an interstitial compartment of the patient through the skin of the patient.

### BACKGROUND

Some patients suffer from chronic pathological conditions in which there is an imbalance of fluids in the body. For example, edema is a medical condition in which fluids flow from the intravascular to the interstitial compartment at a rate that exceeds the removal rate leading to accumulation of excessive fluids in the interstitial and intravascular compartments. Accordingly, edema is an important contributor to heart failure symptoms. Congestive heart failure (CHF), for example, occurs when fluid builds up in the extracellular compartments (intravascular and interstitial). Because of the excess fluid, the heart is unable to pump sufficiently so as to maintain blood flow to meet the body's needs. A person suffering from heart failure may experience shortness of breath, exhaustion, rapid heartbeat, and swollen limbs. Heart failure is a common, potentially fatal condition, and is a leading cause of hospitalization in people over 65 years of age.

In heart failure, the heart works harder to eject blood, leading to a buildup of blood pressure. When elevated blood pressure prevents draining from the interstitial and intravascular compartments of the body, edema, or swelling caused by fluid accumulation in bodily tissues, may occur. The additional work of the heart due to edema weakens the heart, further reducing the ability of the heart to function properly. The fluid accumulation may lead to additional health conditions, hospitalization, and death.

A primary treatment goal in CHF is to achieve adequate fluid and electrolytes balance while avoiding renal dysfunction and other adverse effects. However, even when guideline-recommended therapies are implemented, many chronic heart failure patients still have signs of fluid overload, such as dyspnea and pitting edema.

Current therapies rely primarily on drugs that reduce intravascular volume and pressure, such as diuretics, to reabsorb the excessive interstitial fluids and reduce the edema. With time and the progression of the disease, most of those patients develop resistance to the pharmaceutical treatments, thereby eliminating the primary treatment option. Furthermore, adequate decongestion is not achieved in many patients who rely on current pharmaceutical treatments.

Current medical options are also unable to adequately treat other pathologies that cause a buildup of fluids and electrolytes in the interstitial compartment, such as renal dysfunction, cirrhosis, post operative edema and lymphedema.

### SUMMARY

The present invention provides a system for enhancing removal of excess bodily fluid, electrolytes, and/or nitrogen products (i.e., urea or the like) from a patient's body and through their skin via sweat as a means for treating fluid overload, electrolytic imbalances, and/or urea imbalances. In particular, the present invention includes a fluid stimulation system configured to provide controlled, non-invasive skin fluid transfer, independent and regardless of pharmaceuticals, nutrition, and mental condition of the patient.

The fluid stimulation system includes a therapeutic garment, generally in the form of a robe, that can be worn by a patient. The robe is operably couplable to a console unit configured to control a warm air environment provided via the robe to a patient for causing sweating. For example, the console unit may control at least one of temperature of the environment, flow rate of air into and out of the environment, humidity within the environment, and other additional parameters which ensure safe, as well as effective, operation during use. In some embodiments, for example, the console unit may provide heated air to the robe by way of a heat generator and air blower or fan incorporated into the console unit.

The robe may generally resemble a typical robe, in that the robe has sleeves and is configured to be wrapped around a patient to thereby cover their trunk or torso, majority of their legs, and their upper arms. The robe is designed to cover areas with high concentrations of eccrine sweat glands (trunk, legs, and upper arms), to thereby optimize therapeutic effectiveness, while leaving the neck and head uncovered and protected from airflow to maintain comfort and improve heat tolerance. The robe is further designed to facilitate easy wearing and removal, especially for individuals with limited mobility or disabilities. In particular, the robe can be wrapped around the body and secured with fasteners (e.g., hook-and-loop or the like), thereby reducing effort and increasing accessibility. As such, the robe may provide an ergonomic fit, in that strategically-placed fasteners can provide a secure, adjustable fit, while maintaining a small gap between the robe and a patient's skin for treatment efficiency. Furthermore, the robe incorporates adjustable or detachable sections, enabling selective exclusion of certain body parts, such as legs with ulcers, to provide targeted coverage and greater flexibility for patient-specific needs. The robe may be constructed from lightweight, 100% polyester knit with TPU lamination and antibacterial treatment, thereby ensuring durability, minimal porosity, and thermal insulation.

The robe is able to create a controlled, homogenous warm air environment at a predetermined volume around the patient's body in order to create conditions that initiate sweat production optimized per patient ergonomics. More specifically, the robe comprises a network of air channels through which heated air (provided via the console unit) flows to provide a warm air environment within the interior of the robe (to which a patient is exposed). In particular, the robe includes an inlet port configured to be operably coupled to the console unit via a hose. The network of air channels extending from the inlet port are configured to receive air from the console unit during a treatment procedure. Openings at the sleeves and at the bottom of the robe further enhance airflow, promoting comfort and sweat evaporation without the need for designated outlet ports. The air channels include strategically placed dispersion holes to ensure uniform warm air distribution and efficient sweat evaporation. Furthermore, the network of air channels is arranged so as to enable the warming of specific sections, such as the right or left sides of the robe, to thereby allow for the delivery targeted therapeutic treatment as needed, enhancing flexibility and effectiveness. The network of air channels are constructed to allow for optimized air volume, reduced pressure and noise, while some channels act as insulators (i.e., some channels act as non-dispersing channels) to further improve temperature retention and overall efficiency. The air channels are configured to adjust in size (i.e., width) upon receipt of warm air, allowing for the channels to fit closely to the patient's body, minimizing any gaps and further enhancing skin temperature elevation.

The robe may further include a spacer assembly, which may include a plurality of foam spacer inserts or a three-dimensional (3D) spacer fabric material, that assist in preventing an air channel from collapsing during use. In particular, the spacer assembly may generally be positioned between inner and outer layers of the robe and configured to prevent the inner and outer layers from contacting one another. For example, the spacer assembly may be positioned on a rear portion of the robe and prevents collapsing of air channels at the rear when outer and inner layers would generally be forced upon one another, such as when the patient is lying in a supine position or seated. Accordingly, the spacer assembly ensures consistent airflow within the channels during use, regardless of the patient's position.

The robe is further designed so as to enable sufficient flow of air through the holes and capture the air between the wearable and the skin so that the pressure between the patient's skin and robe will be maintained between 0.5 mmhg-3 mmhg. This pressure enables the expansion of the robe away from the skin to distances of up to 5 cm that enables free flow of air throughout all parts thus enabling evaporation and its subsequent cooling of the patient's core temperature.

The features that enable the pressure and control the inflation of the robe away from the skin are:
1. The configuration of the tunnels are such that when expanded by the airflow in pressures that are between 0.5 mmHg-2 mmHg they shorten in length mostly around the legs.
2. In turn, when the air flows out from the tunnel's holes and onto the patient skin the shortening of the tunnels keep the robe close enough to the patients skin to enable a pressure of 0.5 mmHg that expands the wearable slightly this enabling free flow of air out of the robe thus evaporating all the sweat keeping the patient dry and with normal core temperature

Throughout the treatment, fluids and components of such fluids from the interstitial compartment are removed from the body by the sweat glands, thereby decongesting fluid overloaded patients as well as treating other pathologies. In particular, sweating is one way the body releases fluids. It should be noted that the system of the present disclosure may also enhance fluid transfer through the skin, not only by way of the sweat glands, but also via hair follicles and through the skin itself (e.g., via the epidermis and stratum corneum).

The fluid stimulation system may further include multiple sensors that can be used in controlling the output of heated air to the robe, as well as used in the continuous monitoring of the patient during a given treatment session, including monitoring of patient vital signs. For example, the robe may be equipped with multiple humidity sensors strategically placed in different locations, enabling the measurement of sweat rate. More specifically, the sweat rate may be determined by comparing the relative humidity of the drier air entering the robe with the more humid air within the environment created between the robe and the patient.

Furthermore, the system may include one or more skin temperature sensors placed relative and in proximity to the patient's body (i.e., either in direct contact or indirect contact, such as an infrared sensor) for monitoring the skin temperature and used in verifying that the skin temperature does not, in any circumstance, elevate above a threshold value. The console unit may also include various sensors, including a temperature sensor, a flow meter (or flow sensor), and other sensors.

The sensors of the robe (i.e., humidity sensors) and the console unit provide data to a controller, which is configured to process and analyze such data for controlling and monitoring conditions of the robe. For example, during treatment, data measurements provided by the various sensors may be provided to the controller, which, in turn, is able to control the output of the console unit. For example, in the event that the skin temperature elevates above the threshold value, the controller terminates operation of the console unit, and thus prevents warm air from entering the robe. In some embodiments, the controller processes relative humidity measurements and is able to continuously calculate a sweat rate of the patient during the procedure based on said measurements. Depending on the given treatment, including the specific parameters of the treatment (i.e., planned fluid removal amount and overall time for procedure), the controller is able to control output from the console unit on-the-fly based on sweat weight calculations, including adjusting flow rate and/or inflow air temperature to the robe until a sufficient sweat rate is achieved.

Accordingly, the fluid stimulation system of the present invention, including the unique therapeutic garment (the robe) provides a non-invasive, multiple use device for removing excess fluid in a patient by using external stimulation to increase sweat rates. The robe is able to provide a homogeneous warm temperature environment around a portion of the patient's body as a means of increasing skin temperature and initiating perspiration. The robe, in combination with the console unit, is configured in such a way to ensure that the body core temperature of the patient remains within normal range. In addition, because patient's comfort is paramount with this form of therapy, the sweat evaporates instantaneously, thus avoiding the awareness of perspiration by the patient and enabling long durations of treatments, if required. Furthermore, the robe design allows for use outside of a hospital, including use at an outpatient clinic or even at home, thereby increasing the ease with which treatments can be performed.

The robe operates on the principle of utilizing the natural cooling effect of sweat evaporation to regulate the user's skin temperature. As warm air flows through the air channels of the robe, it elevates the skin temperature, prompting the body to generate sweat. This sweat then evaporates from the skin's surface, and during this process, it absorbs heat from the skin, effectively cooling the body down. The evaporation of sweat draws the excess heat away from the skin, creating a cooling effect that helps maintain a comfortable body temperature. As the skin cools, the process re-stimulates, repeating the cycle of sweat production and evaporation, continuously regulating the user's temperature.

### BRIEF DESCRIPTION OF THE DRAWINGS

Features and advantages of the claimed subject matter will be apparent from the following detailed description of embodiments consistent therewith, which description should be considered with reference to the accompanying drawings.

FIGS. 1A and 1B are diagrammatic illustrations of a system for treating fluid overload conditions of a patient using a fluid stimulation system according to some embodiments of the present disclosure.

FIGS. 2A and 2B are perspective views of a therapeutic garment, generally in the form of a robe, in which patients are wearing an exemplary embodiment of the robe which can be operably coupled to a console unit (FIG. 2B) configured to provide heated air to the robe, which thereby provides a warm air environment at a controlled air volume flow rate around the body of the patient to shift fluids directly and non-invasively from an interstitial compartment of the patient to skin of the patient resulting in controlled fluid loss.

FIG. 3 is an image of an exemplary embodiment of a robe consistent with the present disclosure, illustrating placement of adjustable fasteners.

FIG. 4 are images of a patient securing the robe to their torso via a chest strap.

FIG. 5 is a perspective view of a robe, illustrating the inlet port (to be operably coupled to the console unit via a hose) and the outlets provides at openings in the sleeves and at the bottom of the robe.

FIG. 6 is an image of a portion of the robe illustrating expansion of primary central air channels (of the network of air channels) through which heated air (provided via the console unit) flows to provide a warm air environment within the interior of the robe (to which a patient is exposed).

FIG. 7A is a perspective view of the placement of spacer inserts between inner and outer layers of the robe that assist in preventing an associated air channel from collapsing during use.

FIG. 7B is an image of exemplary spacer inserts in greater detail.

FIGS. 8A and 8B are perspective views of a three-dimensional (3D) spacer fabric material to be positioned between inner and outer layers of the robe that assist in preventing an associated air channel from collapsing during use.

FIGS. 9A and 9B are cross-sectional views of portions of the robe illustrating the network of air channels, illustrating air dispersion holes (FIG. 9B) formed along the inner layer of the robe of some of the channels for allowing for the dispersion of air from the associated channel and into an interior of the robe to thereby create a controlled, homogenous warm air environment around the patient.

FIGS. 10A and 10B are sectional views of side and rear portions, respectively, of the robe illustrating contact surface temperature distribution across the active surface area of the robe as a result of the network of air channels and distribution of air dispersion holes.

FIG. 11 illustrates the change in shape and dimension of the air channels upon receipt of air flowing therethrough, specifically illustrating an increase in channel height (or depth) and a decrease in width, thereby allowing for the channels to conform more closely to the patient's body and minimizing the gap between the robe and a patient's skin, enhancing the efficiency of skin temperature elevation.

FIG. 12 is an image of an exemplary air channel including air dispersion holes strategically placed in the outer thirds of each channel, ensuring that when the channels inflate and air flows toward the patient's skin, the air dispersion holes remain unobstructed by direct skin contact.

FIG. 13 is a sectional view of the robe illustrating pressure distribution relative to a patient's skin as air flows through the network of air channels.

FIG. 14 is a perspective view of another embodiment of a therapeutic garment consistent with the present disclosure.

FIG. 15 is an image of an exemplary construction of the garment, illustrating various layers/components, including a 3D spacer assembly/fabric.

FIG. 16 is a rear view of the garment, illustrating the positioning of 3D spacer assemblies and an edge-guard layer of material provided along the outer boundary of the 3D spacer assemblies.

For a thorough understanding of the present disclosure, reference should be made to the following detailed description, including the appended claims, in connection with the above-described drawings. Although the present disclosure is described in connection with exemplary embodiments, the disclosure is not intended to be limited to the specific forms set forth herein. It is understood that various omissions and substitutions of equivalents are contemplated as circumstances may suggest or render expedient.

### DETAILED DESCRIPTION

Expansion of extracellular volume is central to the pathophysiology of heart failure (HF) and other edematous disorders, resulting in signs and symptoms (edema, dyspnea, orthopnea) commonly referred to as congestion. In ambulatory patients with HF, signs of congestion are strongly related to patient-assessed quality of life and future events. The main reason for hospitalization for worsening HF is related to excess fluid and symptoms of congestion.

Current treatments of congestion rely primarily on diuretics. As stated in a 2019 position statement of the Heart Failure Association of the European Society of Cardiology, “Other than ultrafiltration, the only pathway to get rid of sodium and water is through increased renal natriuresis and diuresis”. Unfortunately, even when fully adhering to these guidelines, adequate decongestion is not achieved in many patients. This leads to a gradual accumulation of fluid and sodium that ultimately leads to overt clinical symptoms. Failure to successfully manage congestion in the outpatient or hospital setting may be related, at least in part, to the limitations of diuretic therapy.

Humans have the capability of producing a large amount of sweat under certain physiological conditions. Eccrine glands are the major sweat glands located throughout the body. The eccrine glands open to the outside world through the sweat pores which produce clear, odorless sweat and are supplied directly from the interstitial compartment. Apocrine sweat glands are another type of sweat gland and are generally found in the armpit, ear, eyelids, and perineum. The secretory portion is larger than that of eccrine glands (making them larger overall). Rather than opening directly onto the surface of the skin, apocrine glands secrete sweat into the pilary canal of the hair follicle.

Sweat glands and sweat rate are influenced by local skin temperature. Sweat may be initiated at skin temperature as low as 33° C. and in a temperature range that varies between 33° C.-39° C. (the upper range value is set to avoid local skin burns). It is expected that a patient may excrete over 100 ml/h of sweat just from local elevation of skin temperature.

This mode of increased sweat rate by elevating skin temperature may be beneficial with edematous patients as the sweat fluids flow from the interstitial compartment, being the source of the edema. In these patients, the clinical need (over 100 ml/h) can be achieved by exposing portions of the patient's body to elevated temperatures that will elevate the skin temperature to levels above 33° C. and below 40° C., and more preferably no greater than 39° C.

The present invention recognizes the advantages of increased sweat rate for treating fluid overload. In particular, the fluid stimulation system of the present invention enhances fluid transfer through the skin, by increased sweat rate, by providing controlled stimulation of fluid transfer directly and non-invasively from an interstitial compartment of the patient through the skin of the patient.

The fluid stimulation system includes a therapeutic garment, generally in the form of a robe, that can be worn by a patient. The robe is operably couplable to a console unit configured to control a warm air environment provided via the robe to a patient for causing sweating. For example, the console unit may control at least one of temperature of the environment, flow rate of air into and out of the environment, humidity within the environment, and other additional parameters which assure safe, as well as effective, operation during use. The robe may generally resemble a typical robe, in that the robe has sleeves and is configured to be wrapped around a patient to thereby cover their trunk or torso, majority of their legs, and their upper arms. The robe is designed to cover areas with high concentrations of eccrine sweat glands (trunk, legs, and upper arms), to thereby optimize therapeutic effectiveness, while leaving the neck and head uncovered for comfort.

The robe is able to create a controlled, homogenous warm air environment at a predetermined volume around the patient's body in order to create conditions that initiate sweat production. Throughout the treatment, fluids from the interstitial compartment are removed from the body by the eccrine and/or apocrine sweat glands, thereby decongesting fluid overloaded patients.

Systems of the invention include a home-use or an outpatient clinic device for chronic patients at a risk of developing fluid overload. The robe is easily adjustable between treatment areas, is easy to operate and monitor, and is easy to clean and maintain.

The invention allows patients to ensure no skin or other heat injuries occur during the treatment and there are no excessive losses of electrolytes or salts that cannot be reabsorbed or digested back. Moreover, sweating is a process that most patients typically have experienced, and adverse effects to the skin are unlikely, even in severe heart failure patients.

Fluid transfer from the interstitial and intravascular compartment can be stimulated and enhanced via sweat, osmosis, other fluid transfer, or a combination thereof using local elevation in skin temperatures or increase in ambient osmotic pressure favoring fluid transfer from the interstitial compartment to outside the body.

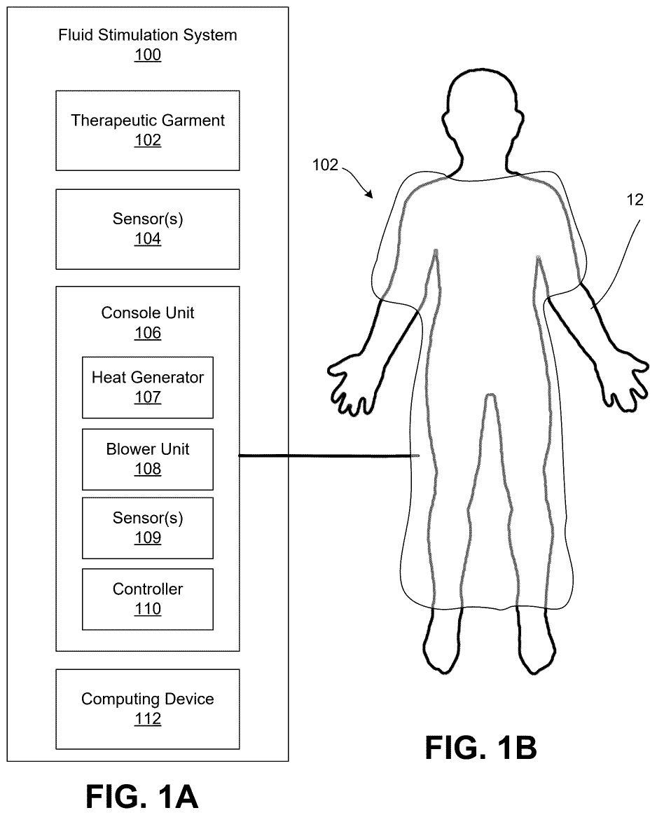

FIGS. 1A and 1B are diagrammatic illustrations of a fluid stimulation system 100 for treating fluid overload conditions of a patient 12. The system 100 generally includes a wearable garment 102 and a console unit 106 to which the garment 102 is to be operably connected.

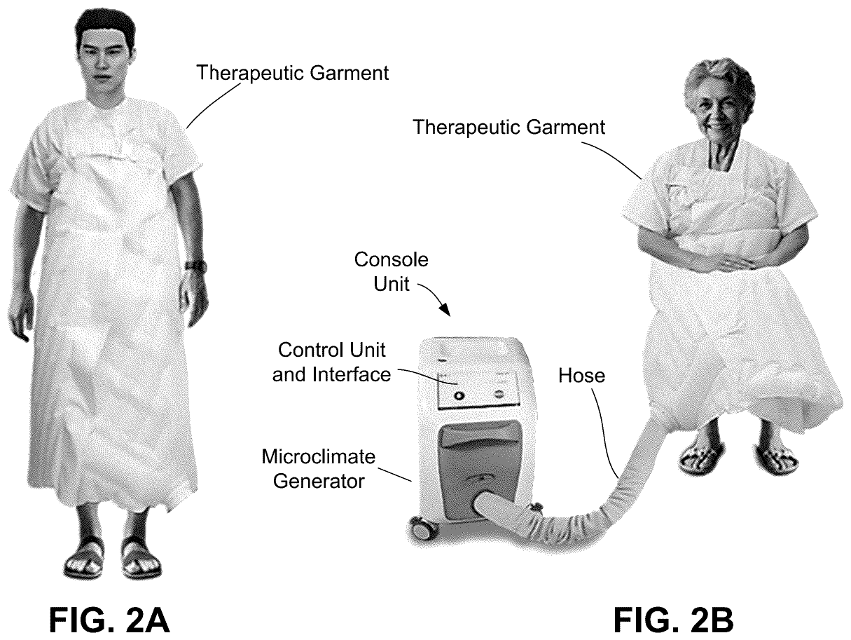

FIGS. 2A and 2B are perspective views of the therapeutic garment, generally in the form of a robe, in which patients are wearing an exemplary embodiment of the robe which can be operably coupled to the console unit (FIG. 2B) configured to provide heated air to the robe.

As illustrated, the wearable garment 102 is generally in the form of a robe. The robe is operably couplable to the console unit 106, which is configured to control a warm air environment provided via the robe to a patient for causing sweating. For example, the console unit 106 may control at least one of temperature of the environment, flow rate of air into and out of the environment, humidity within the environment, and other additional parameters which assure safe, as well as effective, operation during use.

For example, robe may be operably coupled to a console unit 106 via a hose or other connection means through which heated air passed from the console unit 106 can flow into a corresponding inlet port on the robe. For example, a flexible PVC hose may be used. The hose may be releasably connected, at one end, to the inlet port of the robe and releasably connected, at an opposing end, to an outlet port of the console unit 106. For example, each end of the hose may include a snap connector mechanism configured to provide sufficient sealing and quick disconnection to and from the robe and console unit 106.

The console unit 106 is configured to provide heated air to the robe. In particular, the console unit 106 may include a heat generator 107 for heating air and a blower unit 108 for blowing the heated air out of the outlet port of the console unit 106 through the hose and into the robe.

The fluid stimulation system 100 further includes multiple sensors (104, 109) that can be used in controlling the output of heated air to the robe, as well as used in the continuous monitoring of the patient 12 during a given treatment session, including monitoring of patient vital signs. For example, humidity sensors 104 may be placed within an air flow path of the robe. In particular, the robe may include an inlet port (e.g., for receiving heated air from the console unit 106 via a hose or other connection means). The inlet ports may generally be coupled to a framework of channels or chambers of the robe through which the heated air flows to provide the warm air environment within the interior of the robe (to which a patient is exposed). In some embodiments, the inlet port may include a humidity sensor 104 for monitoring the humidity reading of incoming air. The robe may include one or more additional humidity sensors incorporated into portions thereof for monitoring the humidity of the warm air environment to which the patient is exposed during treatment.

Furthermore, the system 100 may include one or more skin temperature sensors 104 placed in contact with the skin of the patient for monitoring the skin temperature and used in verifying that the skin temperature does not, in any circumstance, elevate above a threshold value (i.e., less than 40° C., and more preferably no greater than 39° C.). The console unit 106 may also include various sensors 109, including a temperature sensor, a flow meter (or flow sensor), and other sensors.

The sensors of the robe and the console unit 106 provide data to a controller 110, which is configured to process and analyze such data for controlling and monitoring conditions of the robe. For example, during treatment, data measurements provided by the various sensors may be provided to the controller 110, which, in turn, is able to control the output of the console unit 106. For example, in the event that the skin temperature elevates above the threshold value (i.e., the maximum temperature at which the treatment can be safely performed, which could be as high as 39° C.), the controller 110 may terminate operation of the console unit 106, and thus prevents warm air from entering the robe. In some embodiments, the controller 110 processes humidity measurements collected via the humidity sensors and is able to continuously calculate a sweat rate of the patient during the procedure.

Depending on the given treatment, including the specific parameters of the treatment (i.e., planned fluid removal amount and overall time for procedure), the controller 110 is able to control output from the console unit on-the-fly based on sweat weight calculations, including adjusting flow rate and/or inflow air temperature to the robe until a sufficient sweat rate is achieved.

The fluid stimulation system, most notably the console unit 106, including the heat generator 107, blower unit 108, and controller 110, may generally be controlled via a software-based application running either as a web-based app or running as a local app downloaded on a user's computing device 112. For example, a user (i.e., physician or other medical professional) may utilize a personal computing device 112 (e.g., laptop, tablet, smartphone, etc.) to access software associated with the fluid stimulation device, in which a user interface is provided allowing for the monitoring of a given procedure, as well as subsequent control of such procedure. The computing device 112 is configured to communicate and exchange data with the console unit and controller via a wired or wireless connection (i.e., Wi-Fi network, Bluetooth radio (including Bluetooth Low Energy), Near Field Communication (NFC), etc.). The computing device 112 may also be configured to communicate with other sensors not incorporated into either of the robe or console unit, including sensors for monitoring patient vital signs (i.e., heart rate, blood pressure, saturation, etc.).

Accordingly, the user interface provided on a user's computing device may provide real-time data during the procedure, including, but not limited to, information associated with a current treatment procedure (i.e., elapsed time since treatment began, sensor measurement data and related calculations of the environment provided by the robe, including air temperature and absolute humidity, skin temperature, current sweat rate, accumulative sweat measurement, and more). Furthermore, the user interface may provide a user with input controls for controlling the given treatment, including basic start and stop controls, controls for selecting as well as control over output from the console unit 106 (i.e., air flow rate and heat output).

The robe may generally resemble a typical robe, in that the robe has sleeves and is configured to be wrapped around a patient to thereby cover their trunk or torso, majority of their legs, and their upper arms. The robe is designed to cover areas with high concentrations of eccrine sweat glands (trunk, legs, and upper arms), to thereby optimize therapeutic effectiveness, while leaving the neck and head uncovered for comfort. The robe is further designed to facilitate easy wearing and removal, especially for individuals with limited mobility or disabilities. In particular, the robe can be wrapped around the body and secured with fasteners (e.g., hook-and-loop or the like), thereby reducing effort and increasing accessibility. As such, the robe may provide an ergonomic fit, in that strategically-placed fasteners can provide a secure, adjustable fit, while maintaining a small gap between the robe and a patient's skin for treatment efficiency. Furthermore, the robe incorporates adjustable or detachable sections, enabling selective exclusion of certain body parts, such as legs with ulcers, to provide targeted coverage and greater flexibility for patient-specific needs. The robe may be constructed from lightweight, 100% polyester knit with TPU lamination and antibacterial treatment, thereby ensuring durability, minimal porosity, and thermal insulation.

In some embodiments, the robe may be constructed from internal and external fabric layers made from different materials. For example, an internal layer may be constructed from a thermally conductive textile e.g., metal-coated textiles, metal fiber textiles. Alternatively, a carbon-based textile may be used to promote efficient heat transfer to the skin. In addition, a moisture-wicking liner may be incorporated on the surface facing the user to help manage perspiration and maintain comfort throughout the treatment. The external layer of the robe may be constructed from a material engineered to reduce heat loss by incorporating: (i) low thermal conductivity (ii) low air permeability to limit convective heat loss, and (iii) a reflective barrier layer e.g., an aluminized film, to reduce radiative loss.

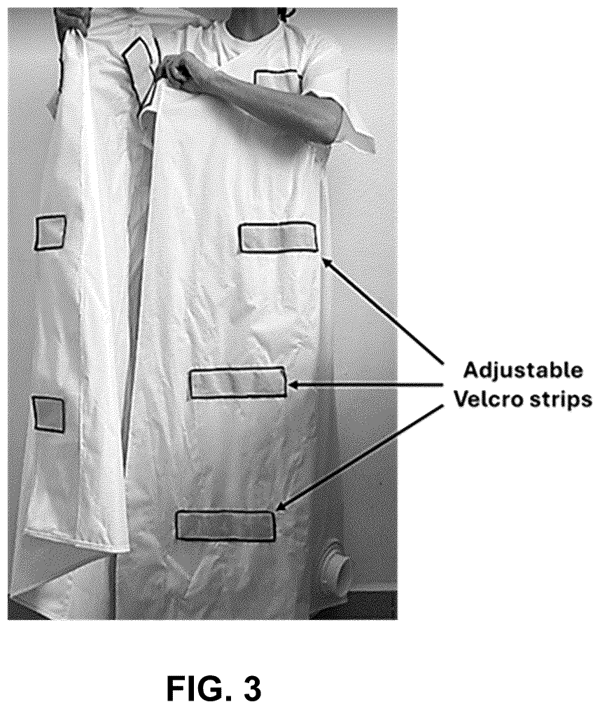

FIG. 3 is an image of an exemplary embodiment of a robe consistent with the present disclosure, illustrating placement of adjustable fasteners. As shown, the robe is designed with ergonomics in mind, featuring an apparatus that enables easy patient-specific adjustments and maintains a small gap between the garment and the skin to optimize treatment efficiency. The robe achieves this ergonomic fit by incorporating horizontal hook-and-loop fasteners, which provide conformability to the patient's body while enabling secure and customizable fit.

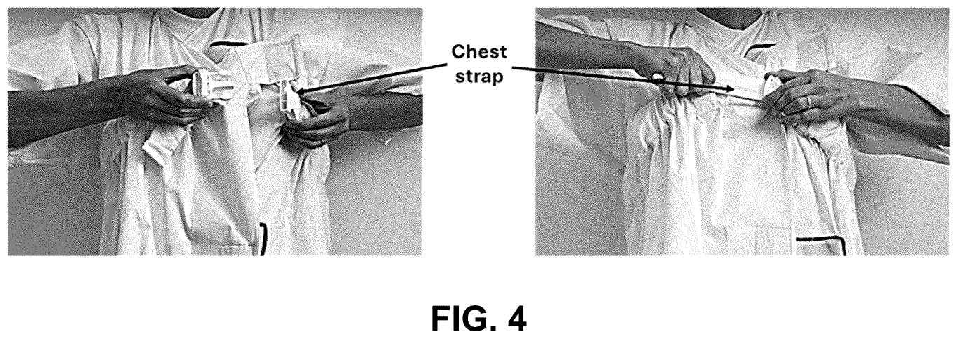

Furthermore, the robe is designed to ensure user comfort by eliminating airflow towards the neck and head. This is achieved through a combination of a chest band and two foam cushions. The chest band prevents excessive airflow in the head and neck area, while the foam cushions, located at the middle of the chest and neck, conform to the body's natural curvature, providing a secure and comfortable fit. FIG. 4 are images of a patient securing the robe to their torso via a chest strap or band.

The desired material for the robe is a lightweight fabric that facilitates the easy inflation of air channels, ensuring smooth airflow by minimizing resistance. Its minimal porosity allows sweat vapor to remain within the garment, enabling more accurate measurement of the sweat rate. Additionally, an optional feature of the material is high thermal insulation on its exterior side, which helps retain warmth within the air channels, maintaining optimal treatment conditions. The robe achieves these desired features by utilizing 100% polyester knit with TPU lamination and an antibacterial treatment for its main fabric.

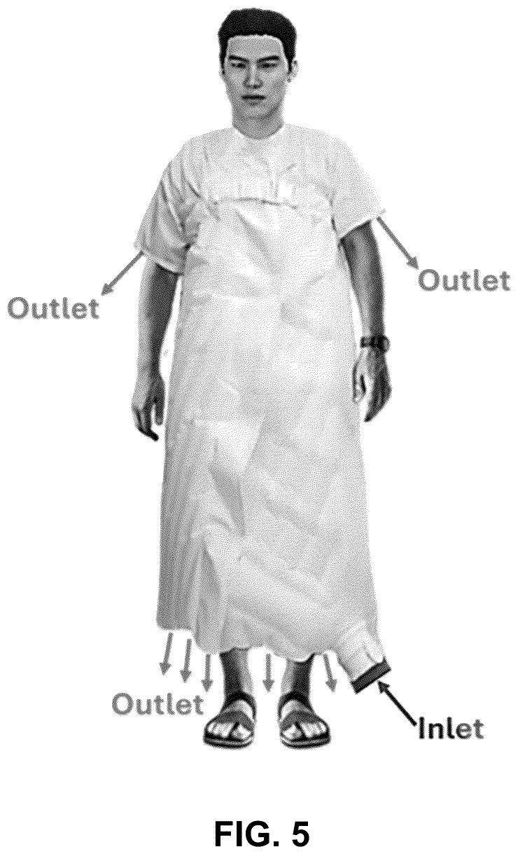

FIG. 5 is a perspective view of a robe, illustrating the inlet port (to be operably coupled to the console unit via a hose) and the outlets provides at openings in the sleeves and at the bottom of the robe. The robe doesn't feature designated outlet ports but relies on the naturally created openings at the sleeves and the bottom for air circulation. These openings play a crucial role in promoting efficient airflow, allowing sweat to evaporate more effectively. This natural air circulation helps maintain a comfortable and dry environment within the interior of the wearable, enhancing user convenience and overall experience.

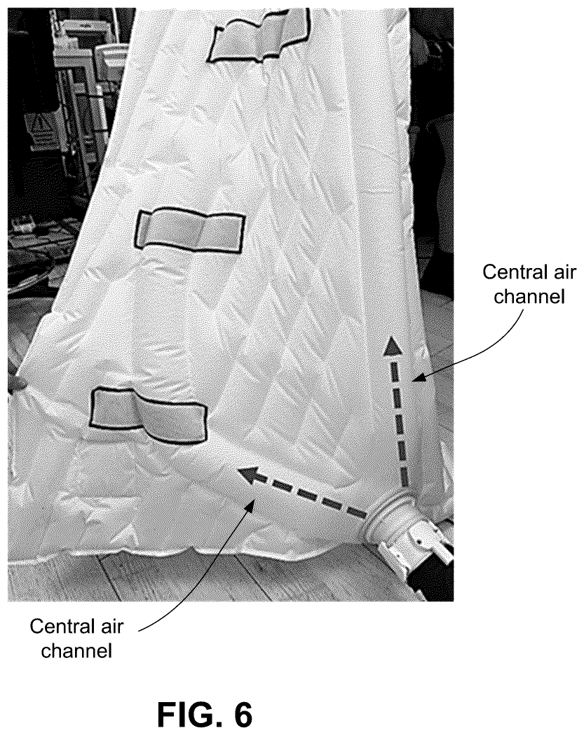

The robe is able to create a controlled, homogenous warm air environment at a predetermined volume around the patient's body in order to create conditions that initiate sweat production. More specifically, the robe comprises a network of air channels through which heated air (provided via the console unit) flows to provide a warm air environment within the interior of the robe (to which a patient is exposed). The network of air channels extend from the inlet port are configured to receive air from the console unit during a treatment procedure. Referring to FIG. 6, an image of a portion of the robe is provided illustrating expansion of primary central air channels (of the network of air channels) through which heated air (provided via the console unit) flows to provide a warm air environment within the interior of the robe (to which a patient is exposed).

As will be described in greater detail herein, the air channels include strategically placed air dispersion holes to ensure uniform warm air distribution and efficient sweat evaporation. Furthermore, the network of air channels is arranged so as to enable the warming of specific sections, such as the right or left sides of the robe, to thereby allow for the delivery targeted therapeutic treatment as needed, enhancing flexibility and effectiveness. The network of air channels are constructed to allow for optimized air volume, reduced pressure, while some channels act as insulators (i.e., some channels act as non-dispersing channels) to further improve temperature retention and overall efficiency. The air channels are configured to adjust in size (i.e., width) upon receipt of warm air, allowing for the channels to fit closely to the patient's body, minimizing any gaps and further enhancing skin temperature elevation.

The robe may further include a spacer assembly, which may include a plurality of foam spacer inserts or a three-dimensional (3D) spacer fabric material, that assist in preventing an air channel from collapsing during use. In particular, the spacer assembly may generally be positioned between inner and outer layers of the robe and configured to prevent the inner and outer layers from contacting one another. For example, the spacer assembly may be positioned on a rear portion of the robe and prevents collapsing of air channels at the rear when outer and inner layers would generally be forced upon one another, such as when the patient is lying in a supine position or seated. Accordingly, the spacer assembly ensures consistent airflow within the channels during use, regardless of the patient's position.

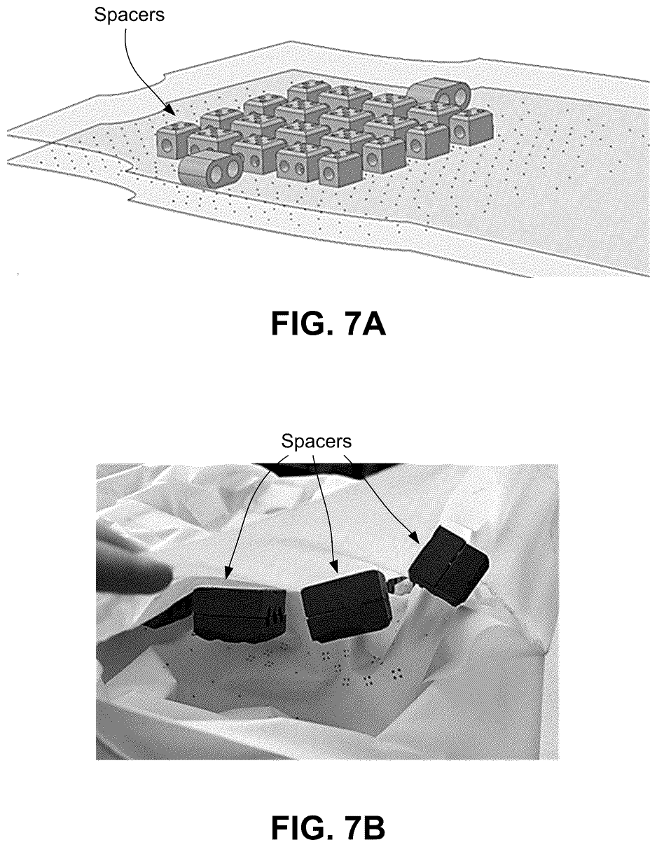

FIG. 7A is a perspective view of the placement of spacer inserts between inner and outer layers of the robe that assist in preventing an associated air channel from collapsing during use. FIG. 7B is an image of exemplary spacer inserts in greater detail. These spacers are typically made of polyethylene foam with holes specifically designed to facilitate efficient airflow.

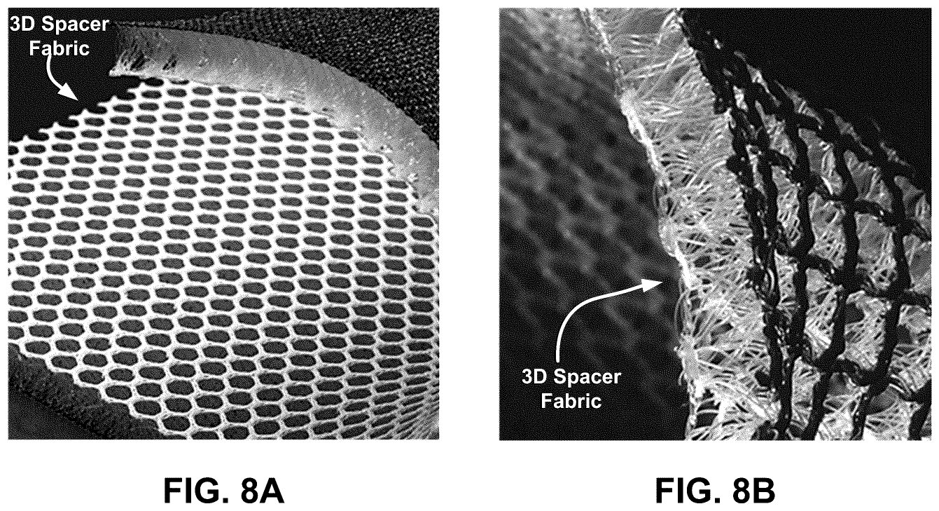

Additionally, or alternatively, the robe may include a 3D spacer fabric material, as shown in FIGS. 8A and 8B, which may allow high air permeability while withstanding high compressive stresses.

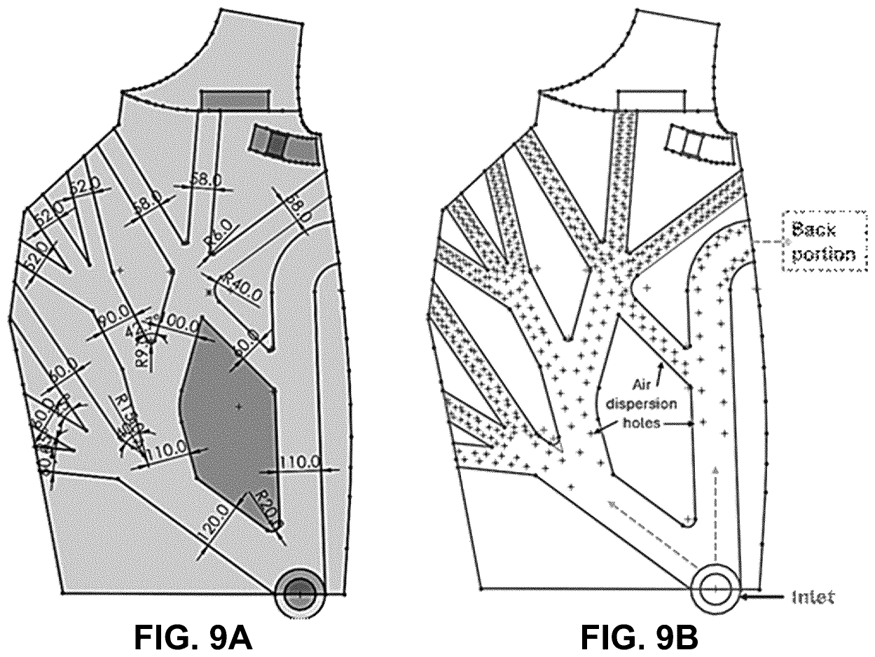

FIGS. 9A and 9B are cross-sectional views of portions of the robe illustrating the network of air channels, illustrating air dispersion holes (FIG. 9B) formed along the inner layer of the robe of some of the channels for allowing for the dispersion of air from the associated channel and into an interior of the robe to thereby create a controlled, homogenous warm air environment around the patient.

There are 2-4 primary channels branching out of the air inlet port, each within a diameter range of 7 to 14 cm). These primary channels are branching out into 2-4 secondary channels in diameter range of 7 to 11 cm). The air channels are designed to minimize areas where a large volume of air can accumulate and potentially cool down. The minimum channel diameter that ensures adequate airflow, even when the channel partially collapses due to fabric wrinkles, is about 5 cm (see FIG. 9A).

Hundreds of air dispersion holes within a diameter range of 0.5-3 cm are positioned along those primary and secondary channels, creating a homogenous air temperature around the user. Air flows through the air inlet at a flowrate range of

$$1. - 2.2 \frac{{m}^{3}}{min}$$

at pressure of approximately 0.5-3 mmHg. One of the primary channels directs the air towards the rear portion of the robe (see FIG. 9B). Between those channels, there are additional channels without air dispersion holes, functioning similarly to a thermos, i.e., the volume of air within those channels act as an insulator 10 as it minimizes contact areas where heat could be transferred to the surroundings, and thus slows down heat transfer processes of conduction and convection. The right panel of the robe functions similarly, as it is designed without air dispersion holes, allowing it to act as a thermal insulator.

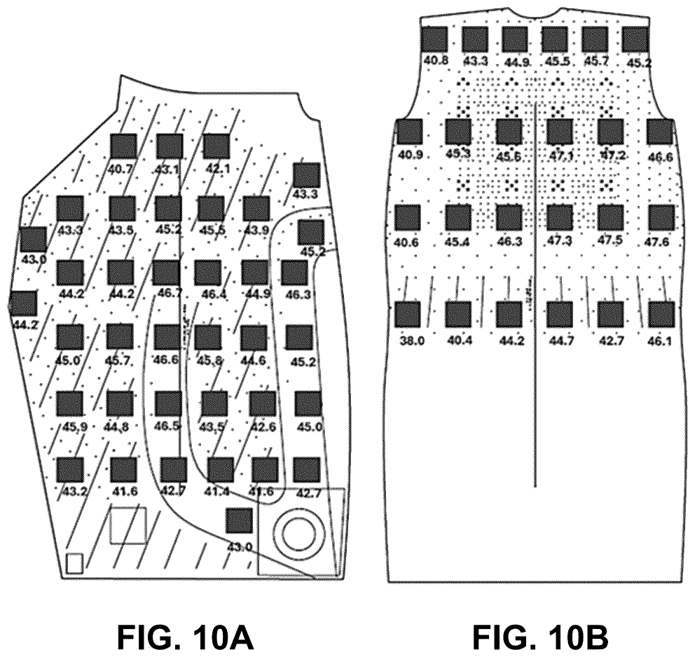

This network of channels ensures a more uniform air temperature distribution across the user's skin. Areas farther from the inlet receive warm air through shorter, direct pathways, resulting in higher temperatures. Moreover, the air dispersion holes are more densely distributed in these distant areas, while the closer regions feature fewer, more widely spaced holes. This design optimizes air flow and enhances temperature balance. FIGS. 10A and 10B are sectional views of side and rear portions, respectively, of the robe illustrating contact surface temperature distribution across the active surface area of the robe as a result of the network of air channels and distribution of air dispersion holes. FIGS. 10A and 10B illustrate contact surface temperature distribution across the active surface area of the robe, featuring air dispersion holes, during system operation at 48° C. with a flow rate of 1.8 m³/min.

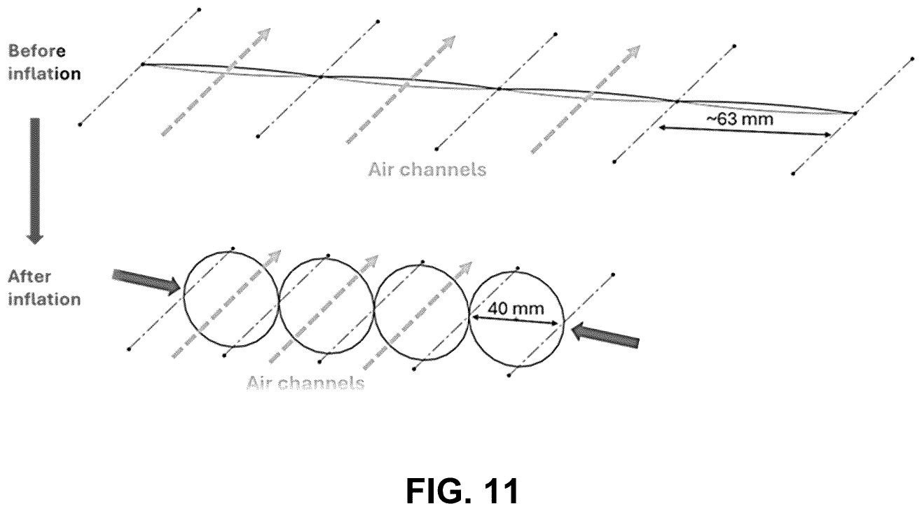

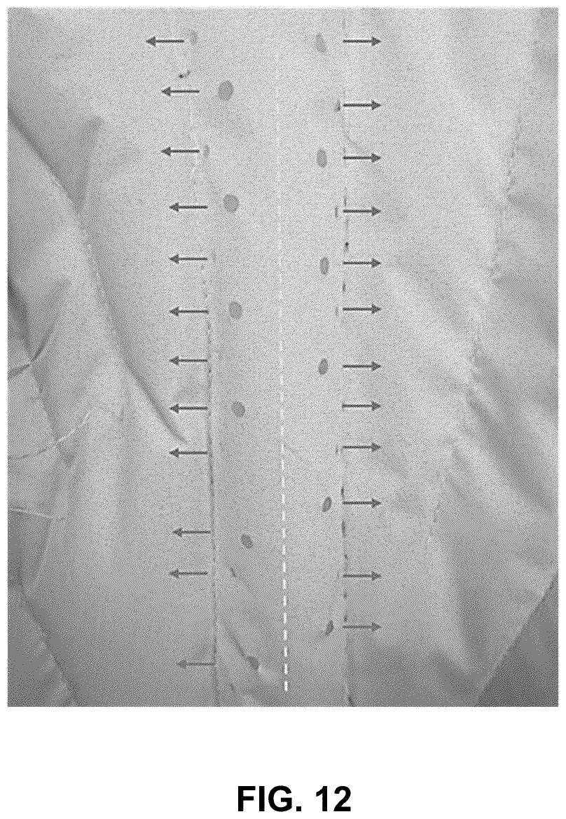

As air flows into the robe and inflates the air channels, their height (or depth) increases while their width decreases to maintain the same air volume within the confined space. FIG. 11 illustrates the change in shape and dimension of the air channels upon receipt of air flowing therethrough, specifically illustrating and increase in channel height (or depth) and a decrease in width, thereby allowing for the channels to conform more closely to the patient's body and minimizing the gap between the robe and a patient's skin, enhancing the efficiency of skin temperature elevation.

FIG. 12 is an image of an exemplary air channel including air dispersion holes strategically placed in the outer thirds of each channel, ensuring that when the channels inflate and air flows toward the patient's skin, the air dispersion holes remain unobstructed by direct skin contact. This positioning directs the airflow to the sides, enhancing efficiency and promoting better air circulation.

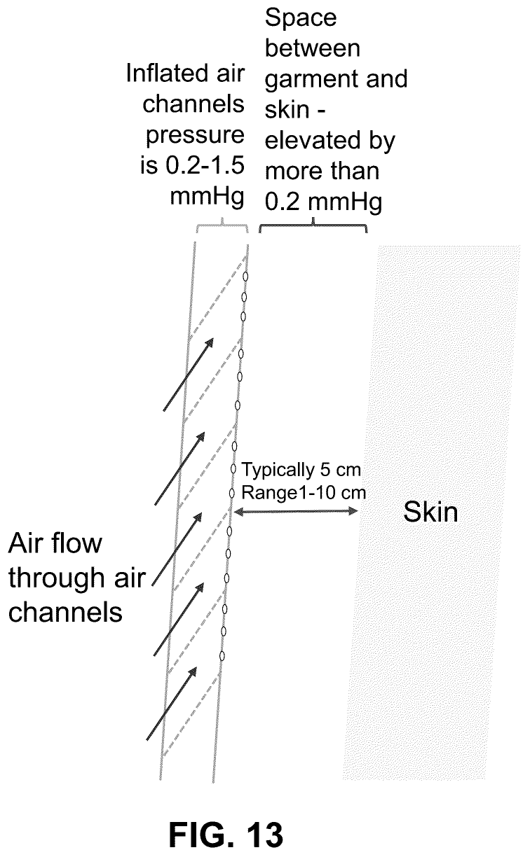

FIG. 13 is a sectional view of the robe illustrating pressure distribution relative to a patient's skin as air flows through the network of air channels.

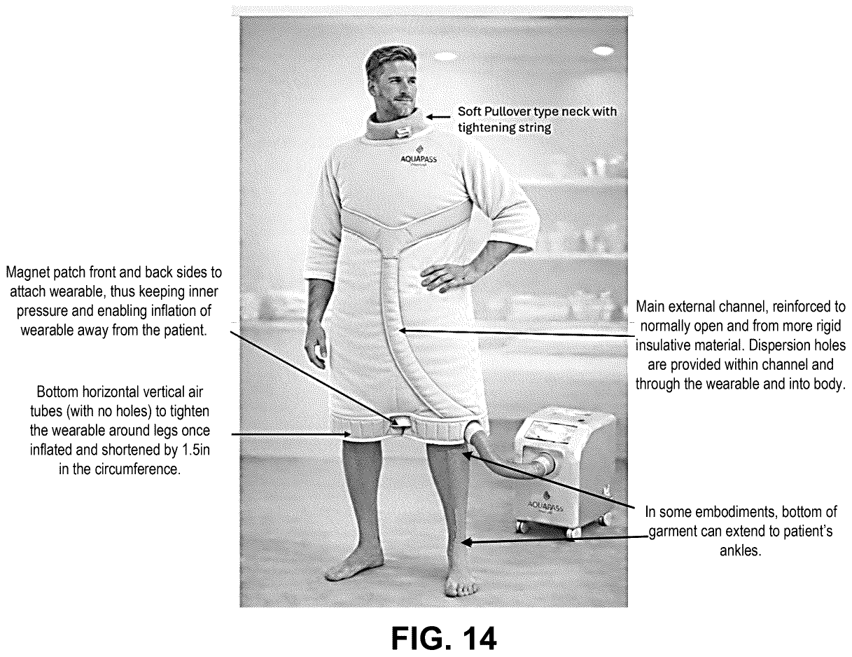

FIG. 14 is a perspective view of another embodiment of a therapeutic garment consistent with the present disclosure. Similar to the embodiment previously described herein, the garment illustrated in FIG. 14 may generally in the form of a robe. The robe is operably couplable to a console unit configured to control a warm air environment provided via the robe to a patient for causing sweating. The robe may generally resemble a typical robe, in that the robe has sleeves and is configured to be wrapped around a patient to thereby cover their trunk or torso, majority of their legs, and their upper arms. The robe is designed to cover areas with high concentrations of eccrine sweat glands (trunk, legs, and upper arms), to thereby optimize therapeutic effectiveness, while leaving the neck and head uncovered for comfort.

The robe is able to create a controlled, homogenous warm air environment at a predetermined volume around the patient's body in order to create conditions that initiate sweat production. Throughout the treatment, fluids from the interstitial compartment are removed from the body by the eccrine and/or apocrine sweat glands, thereby decongesting fluid overloaded patients.

Similar to the wearable garment 102, previously described herein, the robe shown in FIG. 14 is operably couplable to the console unit, which is configured to control a warm air environment provided via the robe to a patient for causing sweating. For example, robe may be operably coupled to a console unit via a hose or other connection means through which heated air passed from the console unit can flow into a corresponding inlet port on the robe.

The robe may generally resemble a typical robe, in that the robe has sleeves and is configured to be wrapped around a patient to thereby cover their trunk or torso, majority of their legs, and their upper arms. The robe is designed to cover areas with high concentrations of eccrine sweat glands (trunk, legs, and upper arms), to thereby optimize therapeutic effectiveness, while leaving the neck and head uncovered for comfort. The robe is further designed to facilitate easy wearing and removal, especially for individuals with limited mobility or disabilities.

The robe is designed to ensure user comfort by eliminating airflow towards the neck and head. For example, the robe may include a pullover-type neck portion, which may include an adjustable tensioning mechanism, such as a drawstring or the like. The robe doesn't feature designated outlet ports but relies on the naturally created openings at the sleeves and the bottom for air circulation. These openings play a crucial role in promoting efficient airflow, allowing sweat to evaporate more effectively. This natural air circulation helps maintain a comfortable and dry environment within the interior of the wearable, enhancing user convenience and overall experience.

The robe is able to create a controlled, homogenous warm air environment at a predetermined volume around the patient's body in order to create conditions that initiate sweat production. More specifically, the robe comprises at least a main channel configured to be coupled directly to the console unit and receive heated air directly therefrom (via a hose or the like). The main channel may be reinforced and rigid to thereby remain in an open state. One or more secondary channels may branch off of the main channel and may be positioned around the robe so as to carry the warm arm to effectively provide a warm air environment within the interior of the robe (to which a patient is exposed). The main channel, and/or the one or more secondary air channels, may include strategically placed air dispersion holes to ensure uniform warm air distribution and efficient sweat evaporation. Furthermore, the air channels (main channel and secondary channel(s)) may be arranged so as to enable the warming of specific sections, such as the right or left sides of the robe, to thereby allow for the delivery targeted therapeutic treatment as needed, enhancing flexibility and effectiveness. The network of air channels are constructed to allow for optimized air volume, reduced pressure, while some channels act as insulators (i.e., some channels act as non-dispersing channels) to further improve temperature retention and overall efficiency. For example, a bottom portion of the robe (at or near the patients lower legs or ankles) may include a plurality of air tubes configured to receive air and inflate (i.e., they do not include air dispersion holes) to thereby assist in tightening the bottom of the robe around the patient's legs.

The robe may further include a spacer assembly, which may include a plurality of foam spacer inserts or a three-dimensional (3D) spacer fabric material, that assist in preventing an air channel from collapsing during use. In particular, the spacer assembly may generally be positioned between inner and outer layers of the robe and configured to prevent the inner and outer layers from contacting one another. For example, the spacer assembly may be positioned on a rear portion of the robe and prevents collapsing of air channels at the rear when outer and inner layers would generally be forced upon one another, such as when the patient is lying in a supine position or seated. Accordingly, the spacer assembly ensures consistent airflow within the channels during use, regardless of the patient's position.

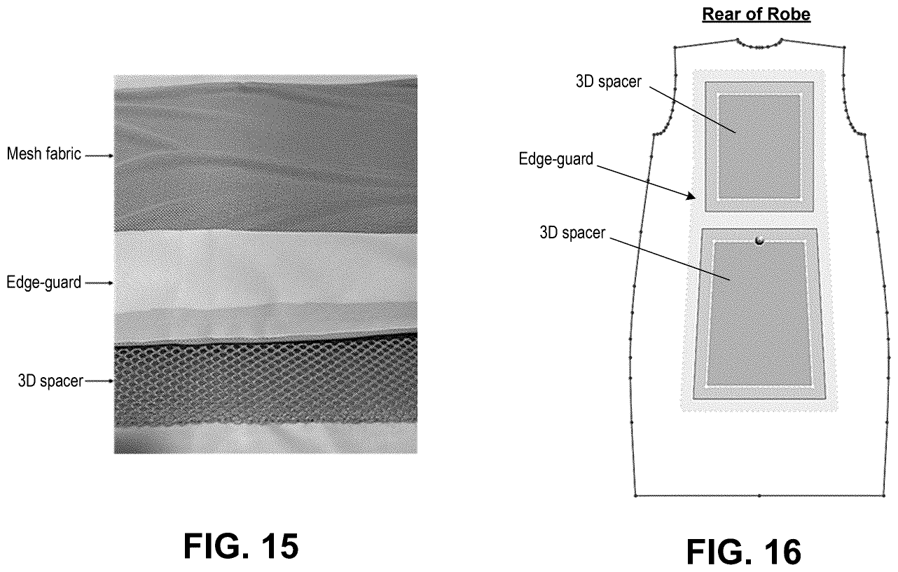

FIG. 15 is an image of an exemplary construction of any embodiment of the garment described herein, illustrating various layers/components, including a 3D spacer assembly/fabric. The robe may incorporate a 3D spacer assembled with a skin-facing mesh fabric. The mesh may provide high air permeability allowing airflow through the spacer to flow toward the skin, thereby promoting efficient air circulation and perspiration management. Alternatively, the inner fabric layer may be highly perforated to promote airflow toward the skin based on the same principles. The 3D spacer may include relatively stiff or sharp monofilaments at its perimeter. To improve user comfort, the robe may incorporate an edge-guard layer along the spacer boundary. This edge guard may be formed from a sealed, puncture-resistant material (e.g., TPU) that prevents monofilament penetration and shields the user from irritation or stinging. FIG. 16 is a rear view of the garment, illustrating the positioning of 3D spacer assemblies and an edge-guard layer of material provided along the outer boundary of the 3D spacer assemblies.

The robe design of FIG. 14 (in combination with the 3D spacer assembly and materials shown in FIGS. 15 and 16) provide advantages, including: 1) the robe inflates away from the body of the patient, thereby enabling improved air circulation and little to no stagnation, which should increase skin temperature and thus evaporation of sweat; 2) the improved neck design enables air flow up to the upper back and arms without risking airflow to the head; 3) the robe may include e only a single layer with no stitches and can be easily inflated away from the body of the patient; 4) less air time in the wearable thereby keeps air temperature and more uniform skin temperature during use; and 5) less pressure within the channels results in less stress placed on the console unit (i.e., the heating and blowing components).

Accordingly, the fluid stimulation system of the present invention, including the unique therapeutic garment (the robe) provides a non-invasive, multiple use device for removing excess fluid in a patient by using external stimulation to increase sweat rates. The robe is able to provide a homogeneous warm temperature environment around a portion of the patient's body as a means of increasing skin temperature and initiating perspiration. The robe, in combination with the console unit, is configured in such a way to ensure that the body core temperature of the patient remains within normal range. In addition, because patient's comfort is paramount with this form of therapy, the sweat evaporates instantaneously, thus avoiding the awareness of perspiration by the patient and enabling long durations of treatments, if required. Furthermore, the robe design allows for use outside of a hospital, including use at an outpatient clinic or even at home, thereby increasing the ease with which treatments can be performed.

The robe operates on the principle of utilizing the natural cooling effect of sweat evaporation to regulate the user's skin temperature. As warm air flows through the air channels of the robe, it elevates the skin temperature, prompting the body to generate sweat. This sweat then evaporates from the skin's surface, and during this process, it absorbs heat from the skin, effectively cooling the body down. The evaporation of sweat draws the excess heat away from the skin, creating a cooling effect that helps maintain a comfortable body temperature. As the skin cools, the process re-stimulates, repeating the cycle of sweat production and evaporation, continuously regulating the user's temperature.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

1. A fluid stimulation system comprising:
a therapeutic garment to provide a warm air environment at a controlled air volume flow rate around one or more body parts of a patient to stimulate sweating and fluid loss in the patient, the garment comprising:
an inlet port configured to be operably coupled to an air fan and heat source and receive warm air therefrom; and
one or more channels extending from the inlet port and configured to receive warm air provided to the inlet port, wherein at least one of the one or more channels comprises a plurality of air dispersion holes through which air, traveling through the at least one channel, can pass to thereby provide uniform distribution of the warm air environment around the one or more body parts of the patient.

2. The system of claim 1, wherein the therapeutic garment comprises a robe-like design.

3. The system of claim 1, wherein the therapeutic garment comprises a pair of sleeves for receiving and covering arms of the patient.

4. The system of claim 1, wherein the therapeutic garment comprises one or more fasteners for securing portions of the garment around respective portions of the patient's body.

5. The system of claim 4, wherein the one or more fasteners comprises hook-and-loop fasteners.

6. The system of claim 4, wherein the one or more fasteners comprises magnetic fasteners.

7. The system of claim 1, wherein the body parts of the patient comprises a torso or trunk, groin, thighs, and upper arms.

8. The system of claim 1, wherein, upon receipt of air, the one or more of the channels expand and increase in height and reduce in width to thereby minimize distance between an inner layer of the therapeutic garment and a surface of the one or more body parts of the patient.

9. The system of claim 1, wherein the therapeutic garment further comprises a spacer assembly provided within one or more of the plurality of channels.

10. The system of claim 9, wherein the spacer assembly comprises a plurality of spacer inserts configured to prevent collapse of an associated channel.

11. The system of claim 10, wherein each of the plurality of spacer inserts comprises a polyethylene foam material and comprises one or more holes for facilitating efficient airflow therethrough.

12. The system of claim 9, wherein the spacer assembly comprises an air permeable three-dimensional (3D) spacer fabric material.

13. The system of claim 1, further comprising one or more channels positioned

14. The system of claim 1, wherein each of the plurality of air dispersion holes has a diameter in the range of about 0.1 cm to about 3.0 cm.

15. The system of claim 1, wherein the one or more channels cooperatively form a network of channels.

16. The system of claim 15, wherein the network of channels comprises a main channel directly connected to and extending from the inlet portion and at least a secondary channel extending from the main channel.

17. The system of claim 15, wherein the network of channels comprises about two to four primary channels directly connected to and extending from the inlet port.

18. The system of claim 17, wherein each of the primary channels has a diameter in the range of about 5 cm to about 14 cm.

19. The system of claim 17, wherein the network of the plurality of channels comprises about 2 two to four secondary channels coupled to and extending from corresponding primary channels.

20. The system of claim 19, wherein each of the secondary channels has a diameter in the range of about 2 cm to about 11 cm.

21. The system of claim 1, wherein the plurality of air dispersion holes are more densely distributed in portions of the one or more channels farther from the inlet port and less densely distributed in portions of the one or more channels closer to the inlet port.

22. The system of claim 1, further comprising one or more sensors for measuring at least humidity of incoming air flowing into the one or more channels and for measuring humidity of air within the warm air environment.

23. The system of claim 1, further comprising a controller communicatively coupled with the one or more sensors and configured to receive humidity measurements therefrom, wherein the controller is configured to adjust temperature and flow rate of incoming air flowing into the one or more channels based, at least in part, on the received humidity measurements.

24. The system of claim 23, wherein the controller is configured to adjust temperature and flow rate of incoming air flowing into the one or more channels to maintain a relative humidity of the warm environment within the therapeutic garment at less than 60% at fluid removal rates between 100 ml/r-600 ml/hr to thereby allow removed fluids on the subject's skin to evaporate and cool the subject's core temperature while avoiding accumulation of fluids within the therapeutic garment.

25. The system of claim 1, wherein the pressure required to inflate the robe and push it away from the skin surface is between 0.2 Hg-1.5 mmHg.

26. The system of claim 1, wherein, when delivering more than 1 cubic meter per minute of air, the therapeutic garment is capable of elevating the pressure between the skin and the therapeutic garment by more than 0.2 mmHg and inflate the therapeutic garment away from the skin, typically about 5 cm and in the range of 1-10 cm, thereby enabling evaporation of the sweating and exchange of air important to maintain relative humidity below 60% at all points.
